(19) United States
(12) Patent Application Publication
(10) Pub. No.: US 2014/0155732 A1
(43) Pub. Date: Jun. 5, 2014

(54) SYSTEMS AND METHODS FOR PORTABLE MAGNETIC RESONANCE MEASUREMENTS OF LUNG PROPERTIES

(71) Applicants: Samuel Patz, Chestnut Hill, MA (US); Mirko Hrovat, Brockton, MA (US); James P. Butler, Brookline, MA (US)

(72) Inventors: Samuel Patz, Chestnut Hill, MA (US); Mirko Hrovat, Brockton, MA (US); James P. Butler, Brookline, MA (US)

(73) Assignee: The Brigham and Women's Hospital, Inc, Boston, MA (US)

(21) Appl. No.: 14/235,337

(22) PCT Filed: Jul. 27, 2012
(86) PCT No.: PCT/US2012/048556

§ 371 Date: Jan. 27, 2014

Related U.S. Application Data
(60) Provisional application No. 61/512,468, filed on Jul. 28, 2011.
(60) Provisional application No. 61/512,714, filed on Jul. 28, 2011.

(30) Foreign Application Priority Data
Jul. 27, 2012 (US) PCT/US12/48556

(51) Int. Cl. G01R 33/34; G01R 33/565; G01R 33/46; A61B 5/08; A61B 5/055; A61B 5/00

(52) U.S. Cl. CPC G01R 33/34092; A61B 5/055; A61B 5/004; G01R 33/46; A61B 5/08; G01R 33/565
USPC 600/410

(57) ABSTRACT

A portable magnetic resonance (MR) system for quantitatively measuring properties of a subject's lungs, such as regional ventilation and lung density, is provided. The portable MR system includes a magnet, radio frequency (RF) coil assembly, and spectrometer system. The magnet can be positioned near the subject's chest. The magnetic field of the magnet substantially homogeneous in a region-of-interest located at a distance from the surface of the magnet that localizes the region-of-interest in the subject's lung. The RF coil assembly includes one or more RF coils that are sized to be positioned near the subject's chest, and receives MR signals from the region-of-interest. The spectrometer system controls the RF coil assembly and computes from the acquired MR signals, a quantitative metric indicative of a characteristic of the subject's lung in the region-of-interest. An active noise cancellation system is provided so RF shielding of the portable MR system is not required.

### CROSS-REFERENCE TO RELATED APPLICATIONS

This application is based on, claims the benefit of, and incorporates herein by reference, U.S. Provisional Patent Application Ser. No. 61/512,468 filed on Jul. 28, 2011, and entitled “Stethoscope,” and U.S. Provisional Patent Application Ser. No. 61/512,714 filed on Jul. 28, 2011, and entitled “Portable Magnetic Resonance Stethoscope to Monitor Pulmonary Edema.”

### STATEMENT REGARDING FEDERALLY SPONSORED RESEARCH

This invention was made with government support under HL100606 awarded by the National Institutes of Health. The government has certain rights in the invention.

### BACKGROUND OF THE INVENTION

The field of the invention is systems and methods for magnetic resonance measurements. More particularly, the invention relates to systems and methods for portable magnetic resonance for regional lung ventilation assessment and lung density monitoring. Further, the invention relates to systems and methods for active noise cancellation in portable magnetic resonance systems.

The assessment of proper and effective ventilatory function in premature newborns suffering from respiratory distress secondary to surfactant deficiency is a difficult task and only crude measures are currently available. In part, this arises from the delicate and fragile nature of premature infants and the life threatening conditions in which they live. Sufficiently high airway pressures necessary to ventilate premature infants are near levels associated with barotraumas, which in itself can be highly detrimental and compromise survival risk Further, because of the time required for a neonate's lungs to mature (weeks to months), inappropriate ventilator settings play a major factor in long term damage due to volume distention or mechanical stretch (volutrauma), continual closing and opening of parenchymal regions (atelectrauma), or ventilator induced pneumonia.

Additionally, in general adult intensive care unit (“ICU”) settings, acute respiratory distress syndrome (“ARDS”) and acute lung injury (“ALI”) are critical problems. ARDS presents with a 30-50% mortality rate. ARDS and ALI are characterized by flooding of the alveoli with fluid, protein, and cellular debris. ARDS is often also characterized by a deficiency of surfactant leading to atelectasis or lung collapse. In such cases, cyclic inflation of the lung by a ventilator translates into cyclic opening and closing of alveoli. In the absence of sufficient surfactant, this collapse and re-expansion with opposing surfaces of alveoli shearing against each other has deleterious and pro-inflammatory effects (described as “atelectrauma”).

In addition to medication and patient positioning, specific ventilator strategies can provide a supportive role for clinical improvement of these conditions. For example, the goal of mechanical ventilation in ARDS is to recruit the lung and maintain its patency throughout the respiratory cycle while producing minimal trauma to lung parenchyma. Currently, however, ventilator adjustment at the bedside is either performed blindly or empirically by adjusting the ventilator to achieve “the best” arterial blood gas measures possible. One significant problem is that determination of success or failure of such adjustments is based on clinical presentation. This often takes sufficiently long that lung injury cannot be reversed. Another significant problem is that blood gases may be within the normal range, but parts of the lung may be over-expanded or collapsed. Either of these conditions can result in permanent damage to those portions of the lung, which will lead to permanently impaired lung function.

The traditional method for evaluating adequate ventilation is X-ray computed tomography (“CT”) scanning. There have been a number of studies using CT for quantifying lung density as a function of lung volume and position with respect to gravity. There are also studies demonstrating the effects of ventilator settings on regional lung density using CT. The problem with using CT, however, is that it cannot be used to frequently evaluate lung patency because of the risk from radiation associated with cumulative exposures. Furthermore, in many cases, patients are too sick to be moved from the ICU to a CT scanner room. For neonates, CT is not an option as neonates are extremely sensitive to any ionizing radiation.

Methods have been presented to quantitatively assess lung ventilation, as well as recruitment, lung distension, and other parameters when changing ventilator settings in intensive care units. Such methods are based on electrical impedance tomography (“EIT”), which is based on applying known variations in current density between a pair of electrodes attached to a subject's chest and detecting changes in voltage, due to impedance changes in the chest, at other pairs of electrodes. Typically, a belt of thirty-two electrodes is applied to the patient's chest to conduct this procedure. Under optimal conditions, EIT can produce an accurate 3D map showing dynamic changes in pulmonary ventilation. However, there are a number of factors that reduce the effectiveness of this technology in real-world conditions. For example, good electrical contact between the electrodes and skin of the subject is necessary and, more importantly, electrode contact resistance must be stable in order to detect longitudinal changes. This is very difficult to achieve when the subject is moving or febrile. There are also several other sources of artifacts besides motion, including skin folds and air pockets.

Although one commercial EIT device has been brought to market, the technology has yet to be adopted for routine clinical use, such as everyday use in the ICU. Furthermore, even if the practical implementation problems are solved for EIT in the pediatric and adult population, it is unlikely that the technology can be translated for use in neonates. For example, the fragility of a neonate's skin as well as the high relative humidity in their environment argue against EIT as an appropriate technology for neonatal intensive care units (“NICUs”). In addition, the very small size of a neonate limits the surface area available for electrode contact and therefore increases the possibility of electrode resistance variation.

Therefore, it would be desirable to provide a noninvasive, portable system for quantitatively measuring ventilation. In addition, it would be desirable to provide such a system that is also capable of measuring other characteristics of the lung, such as lung density. Further, in order to enhance the signal-to-noise ratio (“SNR”) and eliminate the requirement for a radio frequency (“RF”) shielded environment, it would be desirable to provide a system and method for actively cancelling electronic noise, such as electronic noise from environmental sources, in measurements made with such a portable system.

### SUMMARY OF THE INVENTION

The present invention overcomes the aforementioned drawbacks by providing a portable magnetic resonance system for quantitatively measuring characteristics of a subject's lung, such as the degree of ventilation and the lung density.

It is an aspect of the invention to provide a portable magnetic resonance system configured to acquire magnetic resonance signals generated in a region-of-interest in a subject's lung and to calculate therefrom a quantitative metric indicative of a property of the subject's lung. The portable magnetic resonance system includes a magnet, a radio frequency (“RF”) system, and a spectrometer system that is in communication with the RF coil assembly. The magnet is sized to be positioned proximate to the surface of a subject's chest and configured to generate a magnetic field that is substantially homogeneous in a region-of-interest positioned at a distance from a surface of the magnet, in which the distance is sufficiently large so as to position the region-of-interest in the subject's lung. The RF coil assembly includes at least one RF coil sized to be positioned proximate to the surface of the subject's chest and configured to apply an RF field to the region-of-interest and to receive magnetic resonance signals therefrom. The spectrometer system is programmed to direct the RF coil assembly to produce'an RF field in the region-of-interest at a Larmor frequency such that spins resonant with the Larmor frequency in the region-of-interest are excited; direct the RF coil assembly to receive magnetic resonance signals produced in the region-of-interest in response to the applied RF field; and compute from the acquired magnetic resonance signals a quantitative metric indicative of a characteristic of the subject's lung in the region-of-interest.

The foregoing and other aspects and advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1 is a block diagram of an example of a portable magnetic resonance system in accordance with some embodiments of the present invention;

FIG. 2 is an illustration of a magnetic field profile having a substantially homogeneous region a selected distance away from the surface of a magnet, such magnet forming a part of the portable magnetic resonance system of FIG. 1;

FIG. 3 is a pictorial illustration of an example magnet configuration for use with the portable magnetic resonance system of FIG. 1;

FIG. 4 is a pictorial illustration of another example magnet configuration for use with the portable magnetic resonance system of FIG. 1;

FIG. 5 is a plot illustrating the effects of changes in ventilator pressure on lung tissue density, including detrimental pressures leading to either lung distension and barotraumas, or lung collapse and atelectasis;

FIG. 6 is a block diagram of an example of a portable magnetic resonance system configured for use with a hyperpolarized gas contrast agent;

FIG. 7 is a flowchart setting forth the steps of an example of a method for operating a portable magnetic resonance system to obtain quantitative measurements of regional lung properties, such as lung ventilation and lung density;

FIG. 8 is a pictorial illustration of an example of a noise cancelling radio frequency (“RF”) coil for use with the portable magnetic resonance system of FIG. 1 or FIG. 6; and

FIG. 9 is a graph of noise penalty factor as a function of the ratio of correlated random noise to uncorrelated random noise.

### DETAILED DESCRIPTION OF THE INVENTION

A portable magnetic resonance system for measuring a quantitative metric indicative of a property of a subject's lung, such as the degree of regional ventilation or the lung density, is provided. In addition, systems and methods for active noise cancellation that may be used in a portable magnetic resonance system are provided.

It is one aspect of the invention to provide a portable magnetic resonance system capable of measuring lung density and pulmonary edema from magnetic resonance signals acquired from water protons. In this configuration, the portable magnetic resonance system measures the density of tissue and blood in a target region. The density of tissue and blood is approximately one gram per cubic centimeter, and the density of gas is approximately zero. Thus, the density, ρ, within the target region is given by

$$\begin{matrix}ρ = {ρ}_{G} {f}_{G} + {ρ}_{T - B} {f}_{T - B} \\ = 0 \cdot {f}_{G} + 1 \cdot {f}_{T - B} \\ = {f}_{T - B} ;\end{matrix} \qquad ( 1 )$$

where f_{G} is a gas fraction and f_{T-B} is a tissue-blood fraction. Because the two fractions add up to one, when the tissue-blood fraction is determined from the portable magnetic resonance system measurements, the gas fraction can then be determined. Changes in the gas fraction during inhalation or exhalation represent regional ventilation.

It is another aspect of the invention that the portable magnetic resonance system can be used while administering a hyperpolarized gas contrast agent to the patient, such that regional lung ventilation can be directly measured, rather than computed from measurements of lung density. Comparison of the change in hyperpolarized gas concentration from one breath to the next provides information on other pulmonary functional parameters. This configuration is particularly useful for measuring regional ventilation in neonates because the lung volume of neonates is extremely small and the higher signal afforded by hyperpolarized gas compared to hydrogen protons makes the measurement of a regional volume from a neonate's lung feasible.

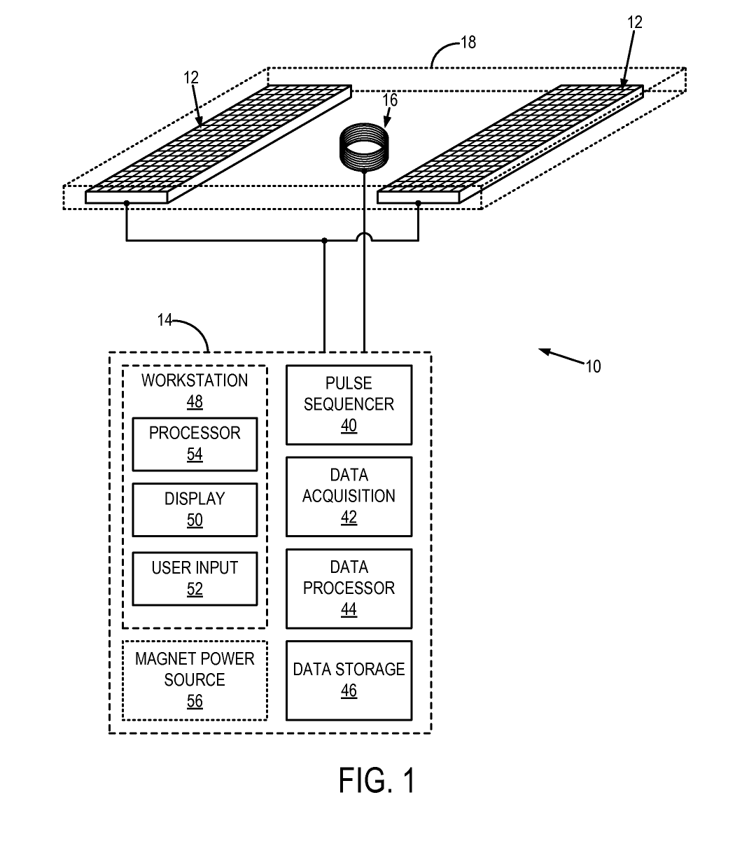

Referring now to FIG. 1, an example of a portable magnetic resonance system 10 that can be used for noninvasive, quantitative measurements of regional lung ventilation and lung density is illustrated. The portable magnetic resonance system 10 generally includes a magnet 12, an electronics subsystem 14, and a radio frequency (“RF”) coil assembly 16. By way of example, the electronics subsystem 14 may include a spectrometer.

In some designs, the magnet 12 and the RF coil assembly 16 can be contained in a single enclosure 18. In these designs, the RF coil may be nested between two permanent magnet poles, thereby fixing the RF coil's position between the magnetic field to provide a well-characterized target region. In use, the enclosure 18 can be positioned in a cushioned layer that lies on top of a traditional hospital bed. The patient can then lie on top of the cushioned layer so that the magnet 12 within the enclosure 18 projects the magnetic field into a region of the patient's lungs. As will be noted below, the magnet 12 and RF coil assembly 16 can also be attached to a mechanical assist device, such as a gantry, that allows precision placement of the magnet 12 and RF coil assembly 16 with respect to the subject's chest while the subject is lying down, sitting, or standing.

As will be discussed below, spatial localization of magnetic resonance signals detected with the portable magnetic resonance system 10 depends on the magnet 12 and the RF coil assembly 16. For example, spatial localization will occur by the intersection of four profiles. The first profile is the magnetic field, B₀, created by the magnet 12. The RF coil assembly 16 includes at least one RF coil, such as an RF coil with a radius, R. The RF coil is responsible for two profiles, the reception profile and the excitation profile, with the RF field falling off along the z-axis as

$${B}_{1} ∝ \frac{1}{{( {R}^{2} + {z}^{2} )}^{3 / 2}} . \qquad ( 2 )$$

The reception profile is proportional to the RF excitation field, B₁, and the excitation profile is given as

sin(γB₁t) (3).

The fourth spatial localization profile is determined by the attenuation produced by water diffusing through the inhomogeneous B₀ field. For a CPMG sequence, the diffusion-related signal attenuation is given by

$${e}^{\frac{t}{{T}_{D}}} ; \qquad ( 4 )$$

where the diffusion time constant, T_{D}, is given by

$${T}_{D} = \frac{3}{{γ}^{2} {G}^{2} D {τ}^{2}} . \qquad ( 5 )$$

Where G is the gradient strength from the inhomogeneous B₀ field, D is the diffusion coefficient, and 22 is the time between 180-degree pulses. For example, a gradient of 0.2 Tesla per meter will give a time constant of T_{D=}0.5 seconds for unbound water. The net effect of this profile is to effectively sharpen the profile created by the magnetic field B₀ because G increases with distance away from the central position of the homogeneous field region. In addition to the localization obtained from the four profiles, the spatial localization profile can further be varied effectively during post processing. Because the Larmor frequency is proportional to the magnetic field strength, B₀, selection of the bandwidth over which the signal is integrated is analogous to sampling the signal from certain spatial regions. This is equivalent to describing the obtained spectrum as a coarse one dimensional image.

Based on the foregoing discussion, simulations incorporating information about the magnetic field sources can be used to determine and visualize the detection region size, strength, and location in relation to magnets 12 and RF coils for a specific portable magnetic resonance system 10 design. Such simulations can also be used to optimize magnet positioning and designs. For example, in a two dipole magnet design, as shown in FIG. 1, the simulation can be used to determine an optimal separation distance of the magnets 12 to achieve a detection region (specifically, a remote saddle point) that extends about 8 centimeters (“cm”) to about 10 cm from the magnet surfaces. It is also noted that, in terms of magnet design, distributing the permanent magnet material in specific ways will improve homogeneity.

A discussion of the individual components of an example portable magnetic resonance system 10 is now provided. First, the magnet 12 is discussed, followed by the RF coil assembly 16. Then, the electronics subsystem 14 is discussed.

The magnet 12 may be a permanent magnet or an electromagnet. Examples of permanent magnets that may be used include monohedral permanent magnets; planar permanent magnets; permanent magnets arranged as a Helmholtz pair, such as a C-magnet; and an array of permanent magnet elements. Examples of electromagnets that may be used include resistive magnets such as a Helmholtz pair or coils with a ferromagnetic structure. Generally, it is contemplated that the magnet 12 will be more efficient when it is sized such that its thickness is less than its width and length.

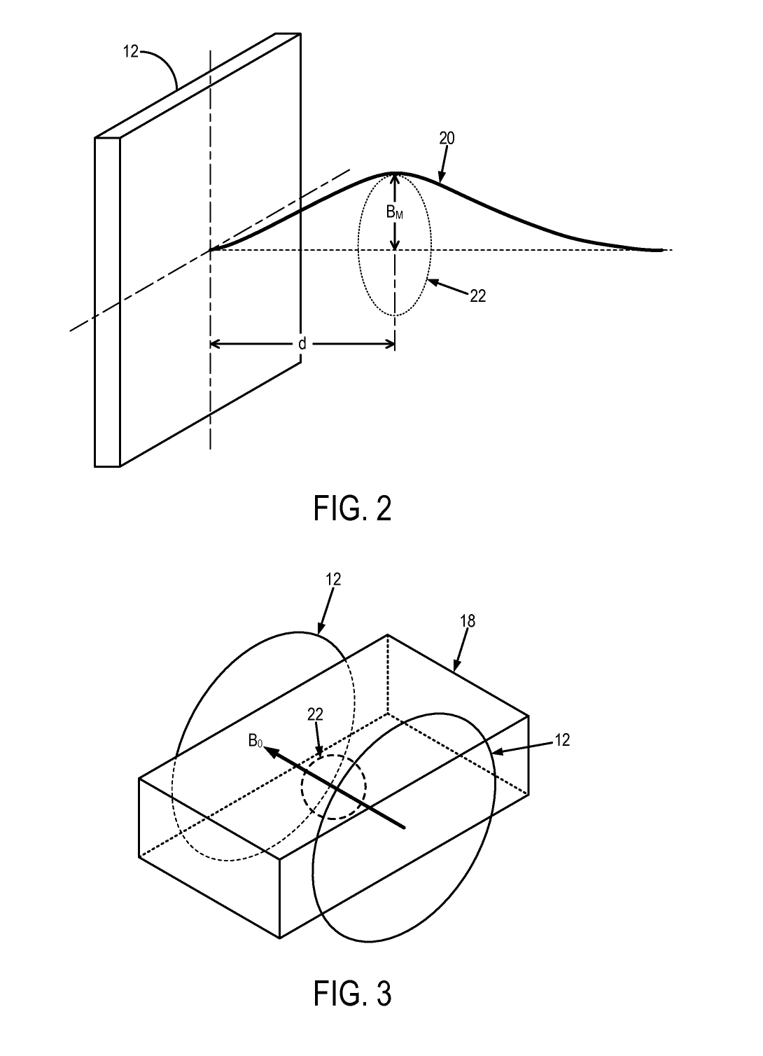

The magnet 12 is preferably designed to generate a magnetic field that is substantially homogeneous in a target region that is remote from the surface of the magnet 12. For example, as illustrated in FIG. 2, the magnet 12 is preferably designed to have a magnetic field profile 20 that has a substantially homogenous region 22 that is external to the surface of the magnet 12. For instance, the substantially homogeneous region 22 is located at a depth, d, from the surface of the magnet 12. With this design consideration, the magnet 12 can be positioned relative to the subject such that the region 22 in which the external magnetic field is substantially homogenous projects into a user-selectable region of the subject's lung. In some configurations, the magnet 12 may be coupled to a mechanically assisted gantry device for selectively moving the magnet 12 over selected lung regions of the subject. Examples of selected lung regions include the apex, base, and middle of each lung, at an approximate depth of about 8 cm to about 10 cm into the subject's chest (that is, within the lung parenchyma).

Preferably, the magnet 12 is a permanent magnet because a permanent magnet does not require a separate power source, can make use of a smaller electronics subsystem 14, can be implemented with a smaller physical size, and has no cooling requirements. When the magnet 12 is a permanent magnet, it may be beneficial to keep the physical size of the magnet small so that the stray magnetic field footprint of the magnet 12 can be significantly localized. This design consideration is especially beneficial for when the portable magnetic resonance system 10 is to be operated in a NICU or ICU setting.

Because most powerful permanent magnets are made from composites that exhibit significant temperature variation for the magnetic field, the electronics subsystem 14 may include a temperature controller (not shown) to control such temperature variations. In some permanent magnet configurations, different materials can be used that produce an overall temperature compensation.

By way of example, the magnet 12 may be a permanent magnet that is an array of permanent magnet elements. The configuration of the array of permanent magnet elements is designed to achieve a particular target region of homogeneity at a desired field strength. With the appropriate configuration of the permanent magnet elements, a second order homogeneity, or higher, can be achieved. The permanent magnet may include magnetic dipoles that are oriented in different directions, or in the same direction. This configuration gives another degree of freedom for designing a more homogeneous magnet. Also, tilted dipoles would allow for a smaller overall size of the magnet 12. In addition, it is possible to have a homogeneous region with the two halves of the magnet oriented with anti-parallel dipoles or with parallel dipoles. To improve performance of the magnet 12, ferro-refraction can be incorporated into the design of the magnet, which can be used to improve efficiency and also to reduce the size of the magnet 12 necessary to achieve a desired field strength and region of homogeneity.

By way of another example, the magnet 12 may be a resistive Helmholtz pair, such as the Helmholtz pair shown in FIG. 3. In this Helmholtz pair configuration, the magnet 12 can be positioned around the subject such that the substantially homogenous region 22 of the magnetic field is positioned in the target region of the subject. Using resistive magnets or other types of electromagnets has the benefit over permanent magnets that electromagnets can be turned on or off as desired. Thus, when electromagnets are used, the portable magnetic resonance system 10 can include a panic button to allow quick shut down of the magnetic field, similar to the panic button used to induce a quench of a superconducting magnet in a traditional magnetic resonance imaging (“MRI”) system.

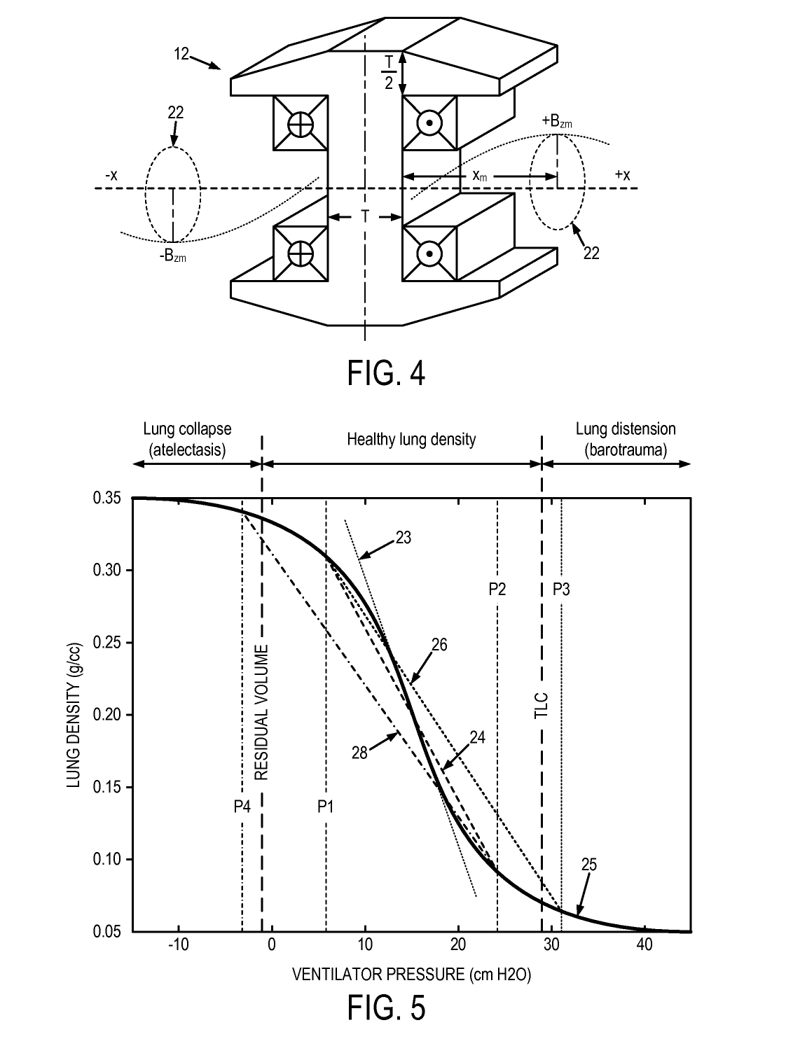

By way of another example, the magnet 12 may be a quadro-ferro-refraction (“QFR”) type of electromagnet, such as the example configuration illustrated in FIG. 4. Using this configuration, the region 22 of substantially homogenous magnetic field, B_{M}, exists at a remote saddle point that is most homogeneous along the x-axis. Although the illustration shows an external B_{M} field on two sides of the magnet 12, during use, one side of the magnet 12 can be shielded. For example, transformer steel can be used to shield one side of the magnet 12 so as to reduce its stray magnetic field footprint. Shielding the magnet 12 in this manner also has the added benefit that ferro-refraction produced by the shielding can result in a smaller magnet or a higher field strength.

The monohedral nature of the QFR design permits its application either as a large magnet 12, or as a small magnet 12. The QFR design is also very efficient due to the generation of image currents in the ferromagnetic material. The QFR magnet design is also applicable to permanent magnets, which can be refined by taking advantage of the ferro-refraction qualities of ferrous materials. Specifically, this ferro-refraction effect may be used to increase the field strength of permanent magnet designs. The addition of ferro-refraction may also reduce the overall weight and size of a monohedral permanent magnet without sacrificing field strength.

The magnet 12 can be a relatively small magnet and, in general, may be designed to produce a low magnetic field as compared to the strength of magnetic fields generated by traditional MRI systems. It is one advantage of the portable magnetic resonance system 10 that a large magnet with a highly homogenous magnetic field, such as is required for traditional MRI systems, is not necessary to quantitatively measure regional lung ventilation, lung density, and other properties of the lung. By way of example, the field strength of the magnet 12 may be 0.1 Tesla (“T”), with a field homogeneity that is less than or equal to ten parts per thousand (“ppt”) at the Larmor frequency of the spin species from which magnetic resonance signals are acquired over a 10 cubic centimeter (“cc”) volume. In some other magnet designs, the magnet field strength may be as low as 50 gauss (“G”) within a 1 cc volume. In yet other designs, that magnet 12 may have a field strength of about 150 G. Furthermore, the magnet 12 can be designed to create a homogenous field region approximately 8 cm to approximately 10 cm away from the magnet's external surface so that the homogenous field region extends into the subject's lungs when the magnet 12 is placed adjacent the subject's chest. These designs with a small magnetic field make it possible to design a magnet that is portable.

Furthermore, using a smaller magnet that may have an inhomogeneous magnetic field profile can be advantageous for the portable magnetic resonance system 10 of the present invention because an inhomogeneous magnetic field profile may be used to spatially localize magnetic resonance signals received by the portable magnetic resonance system 10. This spatial localization capability eliminates the need for gradient coils used with traditional MRI systems, as well as electronics and power requirements of these gradient coils and the necessary considerations that must be taken to account for noise from the gradient system coupling to the RF coil assembly 16. The size of the target region from which measurements of regional lung properties are made can be defined by the field profiles of the magnet 12 and of the RF coil assembly 16.

The maximum size of the target region, referred to as the target field of view (“TFOV”) may be dependent on the design of the magnet 12 and of the RF coil assembly 16, particularly the RF coil or coils that form a part of the RF coil assembly 16. In addition to adjusting the size of the TFOV through the design of the magnet 12 and RF coil assembly 16, the size of the TFOV can also be adjusted during processing of the magnetic resonance signals. For example, magnetic resonance signals may be collected over a large frequency bandwidth and then the volume of the target region may be effectively reduced or selected using frequency filtering with a smaller frequency bandwidth than that used when acquiring the magnetic resonance signals. In some cases, the size of the TFOV can be varied between about 3-10 cc; however, the size of the TFOV can also be smaller than 3 cc or larger than 10 cc depending on magnet and RF coil assembly design, as well as if post-processing is used to adjust the effective size of the TFOV.

The external magnetic field discussed above with reference to FIG. 2 can be achieved using an “open” magnet design, such as a monohedral or planar magnet. This type of magnet design is one-sided and, therefore, can be readily positioned to one side of the subject. The “openness” of the magnet 12 allows for easy access to the subject during a measurement procedure.

It is noted that the magnet 12 may also be designed to include two target regions from which magnetic resonance signals may be acquired, with each target region being positioned at a different depth with respect to the surface of the magnet 12. This design can be achieved, for example, by utilizing three magnets. Some magnet designs also include a symmetrically opposite magnetic field region. While this additional region may be shielded, it can also be used to account for magnetic field drift during use. If signal averaging is employed, temperature drift will shift the magnetic field from the permanent magnet configuration. Thus, by placing a water sample with high SNR at the opposite position of field homogeneity, spectra from this reference sample can be monitored and the spectrum shifted to remove any drift. Signal averages can then be accumulated optimally. A reference sample with known density may also be placed in a symmetrical but identical field strength located outside a patient's chest and used to calibrate the lung density signal measured inside the subject's chest.

With any of the magnet 12 designs discussed above, experimental testing can be used to determine the homogeneous region location and a suitable field strength for acquiring magnetic resonance signals. In this approach, an initial location and field strength can be analytically determined first, and then refined with experimental field mapping.

The RF coil assembly 16 includes, for example, one or more RF coils for transmitting RF energy and for receiving magnetic resonance signals, but may also include an RF power amplifier to drive the RF coil, a preamplifier, and a transmit-receive switch. For example, the RF coil assembly 16 may include a single receive-only RF coil that is concentric with a transmit-only RF coil. As another example, the RF coil assembly 16 may include a receive-only RF coil and an external Helmholtz pair of coils acting as a transmit-only RF coil.

By way of example, short solenoids made with Litz wire may be used in the construction of an RF receive coil to provide optimal detection of magnetic resonance signals from the subject. An example of such a configuration is described in U.S. Pat. No. 5,751,146, which is herein incorporated by reference in its entirety. When the RF coil assembly 16 is operating at lower frequencies, the tuning and matching elements can be located remotely from the RF coils, which allows for a very broadband approach to the electronics.

Active noise cancellation is preferably implemented to eliminate the necessity of operating the portable magnetic resonance system 10 in an RF-shielded room. This approach strongly enhances the portability of the portable magnetic resonance system 10. This approach also considerably simplifies the operation of the device in an ICU, patient bed, or other portable location such as a field hospital or battlefield evacuation vehicle. Examples of active noise cancellation hardware and methods are described below in more detail.

In some configurations, the RF coil assembly 16 may include a mechanically assisted gantry device (not shown) for selectively moving an RF coil over selected lung regions of a subject. As noted above, the magnet 12 may also be coupled to such a gantry such that the magnet 12 and RF coil assembly 16 can move together to select a desired region-of-interest in the subject's lung from which measurements are obtained. In this design, the magnet 12 and RF coil assembly 16 can remain in a fixed spaced relation as the gantry is moved relative to the subject. In other configurations, the magnet 12 and the RF coil assembly 16 may be allowed to move relative to each other as well. In addition, in some configurations, other detection sensors may be used in the RF coil assembly 16, such as superconducting quantum interference devices (“SQUIDs”). It is noted that some of the components of the RF coil assembly 16, such as the amplifiers and transmit/receive switch, may alternately be part of the electronics subsystem 14.

Referring again to FIG. 1, the electronics subsystem 14 can include electronic components similar what is found in a portable magnetic resonance spectrometer. In particular, the electronics subsystem 14 can include a pulse sequence component 40, a data acquisition component 42, a data processing component 44, a data store component 46, and a workstation 48 or computer system having a display 50 and a input 52, such as a keyboard. The workstation 48 includes a processor 54, such as a commercially available programmable machine running a commercially available operating system. The workstation 48 provides the operator interface that enables scan prescriptions to be entered into the ventilation stethoscope system 10. The electronics subsystem 14 can also include a magnet power device 56 for powering the magnet 12 if necessary; for example, when the magnet 12 is an electromagnet.

The pulse sequence component 40 functions in response to instructions downloaded from the workstation 48 to operate the RF coil assembly 16. RF excitation waveforms are applied to the RF coil, or a separate local coil, by the RF coil assembly 16 to perform the prescribed magnetic resonance pulse sequence. Responsive magnetic resonance signals detected by the RF coil, or a separate local coil, are received by the RF coil assembly 16, amplified, demodulated, filtered, and digitized under direction of commands produced by the pulse sequence component. The RF coil assembly 16 also includes an RF transmitter for producing a wide variety of RF pulses used in magnetic resonance pulse sequences. The RF transmitter is responsive to the scan prescription and direction from the pulse sequence component to produce RF pulses of the desired frequency, phase, and pulse amplitude waveform. In one example, the pulse sequence component 40 can utilize spin echo sequences by generating multiple 180 degree excitation pulses, as in a Carr-Purcell-Meiboom-Gill (“CPMG”) sequence. This may increase SNR since, since as the main magnetic field produced by the magnet 12 is inhomogeneous, the effective T2* will be short.

The pulse sequence component 40 also optionally receives patient data, such as respiratory signals, for example, via a physiological acquisition controller (not shown) or a mechanical ventilator (not shown) being used to ventilate the subject. Such signals may be used by the pulse sequence component to synchronize, or “gate,” the performance of the scan with the subject's respiration.

The digitized magnetic resonance signal samples produced by the RF coil assembly 16 are received by the data acquisition component 42. The data acquisition component 42 operates in response to instructions downloaded from the workstation 48 to receive the real-time magnetic resonance data and provide buffer storage, such that no data is lost by data overrun. In some scans, the data acquisition component 42 does little more than pass the acquired magnetic resonance data to the data processing component 44.

The data processing component 44 receives magnetic resonance data from the data acquisition component 42 and processes it in accordance with instructions downloaded from the workstation 48. For example, the magnetic resonance signals may be processed to adjust the effective size of the TFOV, as described above, or to compute quantitative metrics of the subject's lung properties, such as regional lung ventilation and lung density. Processing methods specific to the portable magnetic resonance system 10 are further described below. The output of this processing may be used to inform a physician about how to adjust the ventilator. Alternatively, the ventilator may be in communication with the portable magnetic resonance system 10 to perform a scan of ventilation of lung density as a function of different ventilator settings. In the alternative configuration, the output would then be supplied to a clinician or a computer program for determining the optimal ventilator settings based on the feedback obtained from the portable magnetic resonance system 10.

Calculated quantitative metrics and magnetic resonance signals that are processed by the processing component 44 are conveyed back to the workstation 48 where they are stored. Magnetic resonance signals acquired in real-time may be stored in a database memory cache (not shown), from which they may be output to operator display 50. Magnetic resonance signals may also be stored in a host database on disc storage (not shown). When such signals have been reconstructed and transferred to storage, the data processing component 44 notifies the data store component 46 on the workstation 48. The workstation 48 may be used by an operator to archive the signals or send the signals via a network to other facilities.

By way of example, the electronics subsystem 14, and in particular the pulse sequence component 40, can use a multi-spin echo (such as a CPMG sequence) to acquire magnetic resonance signals from the target region in the subject. Because this sequence utilizes 180-degree pulses that refocus all sources of dephasing, it is an appropriate signal averaging sequence for field regions with low homogeneity.

During use of the portable magnetic resonance system 10, it is contemplated that, for a detection region of about 30 cc and a magnetic field strength of approximately 0.02T, about 20 seconds of data acquisition can achieve a suitable SNR to measure lung density changes. In another example, it is contemplated that, for a detection region of about 25 cc, about 2 minutes of acquisition time will yield an estimated SNR of 220. SNR can be increased by signal averaging over a longer time period (for example, while the subject is free breathing and gating each acquisition to different points in the breathing cycle) or by increasing the volume of the detection region (for example, creating a sphere with a diameter of about 5 cm to about 10 cm). This relatively short data acquisition time (such as between about 20 seconds and about 2 minutes) can allow substantially real-time monitoring of lung density at the target region, despite using a low magnetic field strength. With regard to spatial resolution of such a small volume (such as about 25 cc to about 30 cc), it is noted that lung density changes relatively slowly with position and, as a result, low spatial resolution is adequate for obtaining lung density measurements needed for evaluating lung patency in accordance with the methods described above.

As noted above, the portable magnetic resonance system 10 can be used as a noninvasive device to measure regional ventilation, to evaluate lung function and airway patency, and to measure lung density and/or monitor interstitial pulmonary edema in subjects. In light of the simple design (small magnet, no gradient coils, less electronics, etc.), the portable magnetic resonance system 10 is also a portable, low cost solution for quantitatively assessing the lung. As discussed above, the portable magnetic resonance system 10 can be used in NICU environments, for example to facilitate titration of ventilator settings in infants suffering from respiratory distress. In these environments, the ability to monitor collapsed and atelectatic lung regions, and their response in reopening of units with titration of ventilatory strategies, can vastly improve the medical care necessary for survival, as well as minimize damage inflicted on the pulmonary structures during mechanical ventilation. Furthermore, since the portable magnetic resonance system 10 utilizes the nuclear magnetic resonance phenomenon as the measurement modality, the portable magnetic resonance system 10 is capable of measuring regional ventilation without subjecting neonates to ionizing radiation.

It is also contemplated that the methods for measuring regional ventilation can further be translated toward quantitatively addressing the nature of lung unit reopening as a function of ventilatory strategy. For example, a rational foundation for ventilatory strategies in newborns with respiratory distress syndrome can be built based upon the regional ventilation measurement strategies and other variables such as levels of positive end expiratory pressure (“PEEP”), periodic deep breaths, plateau pressure settings, management of the interaction between ventilatory frequencies and tidal volumes, and so on.

The portable magnetic resonance system 10 can also be applicable in settings other than the NICU. For example, in pediatric intensive care units (“PICUs”) or general intensive care units (“ICUs”), the portable magnetic resonance system 10 can be used as an assessment tool for optimally adjusting ventilator settings to allow proper ventilation and prevent ventilator induced lung injury. For example, in one specific application, the portable magnetic resonance system 10 can be used in an ICU environment to aid clinicians in the care of patients with Acute Lung Injury (“ALI”) and Acute Respiratory Distress Syndrome (“ARDS”). In clinics or doctor's offices, the portable magnetic resonance system 10 can be a helpful tool for measuring regional ventilation in cystic fibrosis patients, providing a more accurate method for assessing the efficacy of treatment; specifically, by measuring regional ventilation before and after treatment. In research settings, the portable magnetic resonance system 10 can be a useful tool to aid in disease research; for example, sickle cell disease and pneumonia research.

In field hospitals, the portable magnetic resonance system 10 can be a helpful tool to assess lung injuries in wounded soldiers. For example, the portable magnetic resonance system 10 may be used to detect a pneumothorax, such as might be needed for a wounded soldier near the battlefield. In general, the presence of a pneumothorax can be detected by measuring lung density as a function of inhalation and exhalation. In a traumatic pneumothorax that occurs from external bullets or shrapnel, the pleural space fills up with air both from the lung and from air entering from the outside of the body through the wound. Hence, most of the thoracic cavity will be filled with air. The collapsed lung will only occupy a very small volume. In addition, if there is a hemothorax, blood will fill the gravitationally dependent part of the lung.

Based on this, the portable magnetic resonance system 10 can be used to detect both a pneumothorax and a hemothorax. A pneumothorax can be detected by interrogating the non-dependent regions of the lung where only air is expected to be in the thoracic cavity. In these regions, a very low density that does not change with breathing would be measured. To detect a hemothorax, the dependent regions of the lung would be interrogated. In these regions, there should be blood in the thoracic cavity; thus, a high density (similar to normal tissue) that does not change with breathing would be measured. In contrast, in healthy lung tissue a lung density that changes during breathing would be measured.

The portable magnetic resonance system 10 is useful for monitoring patient progress by providing a functional measurement that indicates whether or not a particular region of the lung is collapsed/consolidated, filled with fluid, or overdistended. As one example, the portable magnetic resonance system 10 may be useful for determining optimal ventilator parameters to maximize alveolar recruitment without applying too much pressure that would cause alveoli to distend, resulting in damage to the very sensitive alveolar structure. Currently, adjustment of ventilator parameters is performed at the bedside in a substantially blind manner using blood gas measurements; however, this is not directly related to lung patency and, as a result, there exists the possibility of titrating ventilation parameters that can cause harm to the alveoli. Adjustable ventilator parameters include positive end expiration pressure (“PEEP”) and maximum or peak inspiratory pressure (“PIP”).

PEEP is typically a small positive pressure present at the end of expiration that keeps alveoli open at this point in the respiratory cycle. This is an important parameter for ARDS and ALI patients, who lack surfactant on the surface of their alveoli. Surfactant makes it easy for the alveoli to open and close during ventilation cycles. Without surfactant, once an alveolus closes, it takes a significant amount of pressure to reopen it and repeated opening and closing can cause trauma to the lung. In particular, repeated reopening subjects the alveoli to substantial shear forces. These forces act on the delicate alveolar septal walls and, after many ventilatory cycles of closing and opening, has deleterious and pro-inflammatory effects (described as “atelectrauma”).

PIP is the maximum pressure applied by the ventilator during inhalation. If the PIP is set too high, the lung is expanded beyond total lung capacity (“TLC”), exposing pulmonary tissues to excess pressure, which can create overdistension or barotrauma, also damaging the lung over time. Either of these scenarios can easily cause ventilator induced lung injury (“VII”), which can often be fatal.

The effect of other ventilator parameters on ventilation or lung density, such as frequency of breathing, fraction of the respiration cycle that is inhalation versus exhalation, and so on, can also be examined with the portable magnetic resonance system 10 of the present invention.

Referring now to FIG. 5, a plot showing how lung density behaves as a function of pressure applied at the airway opening, that is, as a function of ventilator pressure is shown. The line 23 above the lung density versus pressure curve 25 shows the maximum negative slope of the lung density versus ventilator pressure curve 25. As the pressure increases, air flows into the lung, the lung expands, and the tissue density decreases. Increasing the ventilator pressure can not only expand the volume of gas in alveoli that are already open, but can also recruit alveoli that are essentially closed. As the lung expands with increasing pressure, the TLC volume is reached, which is the maximum volume to which a subject can voluntarily inhale. If the maximum inspiration pressure (PIP) of the ventilator is increased, such that the lung expands beyond TLC, the decrease in lung density with increasing pressure shows diminishing returns as the alveoli reach their elastic limit. The delicate septal tissue of the lung can be damaged if it becomes overdistended. This regime produces injury to the alveolar tissue and is called barotrauma.

At the other end of the breathing cycle, the ventilator pressure is reduced and the patient exhales. The lowest ventilator pressure is the PEEP. Typically the PEEP is not set to zero because in patients with ALI, many of the alveoli are in a collapsed state at zero airway opening pressure and below. The condition of alveolar collapse is called atelectasis. Subjects with ALI do not have sufficient surfactant in their lungs to reduce the shear forces when a collapsed alveolus is forced open; thus, if alveoli are allowed to collapse and then open on each breath, the repetitive shear forces can cause injury.

The portable magnetic resonance system 10 of the present invention is capable of measuring the change in lung density with a change in ventilator pressure. Suppose a ventilator is being operated with a PEEP of P1 and a PIP of P2. The portable magnetic resonance system 10 can be operated to measure the change in lung density between end inhalation (P2) and end expiration (P1). The following parameter can be computed from these values:

$$slope = \frac{Δρ ( P 1 , P 2 )}{P 2 - P 1} ; \qquad ( 6 )$$

where Δρ is the change in lung density between P2 and P1. This parameter measures the slope of line 24. If, however, the PIP is increased to P3 in the desire to open up or recruit more lung, then the ventilator is operating between pressure P1 and P3 and the slope is shown by line 26. Because the slope decreases substantially, it can be shown that the extra pressure has diminishing returns and the ventilator is operating in an unsafe region for the lung where barotrauma may result. On the other hand, if the PEEP is lowered from P1 to P4 with the desire to increase gas exchange with a greater pressure change during the breathing cycle, a decreased slope shown by line 28 would be observed. The decreased slope indicates that the lower PEEP pressure does not produce a similar change in lung density per unit change in pressure as before, indicating that some of the alveoli were closing and not responding below a certain pressure. With this evidence of potential atelectrauma, this change in PEEP would be rejected.

It is noted that, although lung density can be determined through CT imaging, the magnetic resonance density monitor can be used in an ICU environment and can be used to frequently or continuously evaluate lung patency without the risk from radiation associated with cumulative exposures (as is the case for CT imaging). Thus, the portable magnetic resonance system 10 can provide substantially real-time, easy to interpret data in a safe manner at the bedside of in the ICU for use in optimizing ventilation parameters. Furthermore, providing frequent or continuous ventilation optimization may provide a solution to reduce mortality related to conditions such as ARDS and ALI. It can also provide a solution for diagnosing a pneumothorax in a wounded soldier close to the battlefield, thereby also reducing mortality.

It is also noted that lung density measurements and comparisons can be performed at different regions within the lung in order to measure lung density as a function of gravity. For example, with a subject in the supine position, the weight of the lung on itself causes greater stretching of the alveoli in anterior portions of the chest in comparison to posterior portions. The hydrostatic pressure of blood also contributes to gravitationally dependent lung density. Therefore, optimization of ventilation parameters can also be dependent on body positioning and gravity. Measuring lung density at different regions can be achieved by moving the RF coil assembly 16 and/or the magnet 12 or by adjusting the penetration depth of the magnetic field generated by the magnet 12, as further described below.

Thus, the portable magnetic resonance system 10, in combination with the above methods, can provide a functional measure of how different regions of the lung respond to treatment, allowing a clinician to monitor lung density when adjusting PEEP to make sure that lung density never increases above a maximum value indicating alveolar collapse, and to monitor lung density when increasing PIP to make sure that the lung continues to expand and lung density continues to decrease, without reaching the elastic limit indicating the lung is being over-stretched. The portability of the portable magnetic resonance system 10 can allow for continuous monitoring of these parameters, for example in ICU environments, in order to provide patient-specific titration in real time or near real time. Furthermore, the portability of the portable magnetic resonance system 10 allows for the use of ventilation monitoring in field hospital environments, for example to aid in the treatment of trauma-related ARDS in wounded soldiers (often termed “shock lung”).

As discussed above, the portable magnetic resonance system 10 can also be useful for monitoring interstitial edema at the bedside. Acute pulmonary edema, or excess fluid accumulation in the alveoli or lung parenchyma, can be fatal if not treated quickly. In addition to ARDS and ALI, pulmonary edema can be cardiogenic (in particular, caused by the heart failing to remove fluid from the pulmonary vasculature). Using the portable magnetic resonance system 10 and the above-described methods, proton density measurements in a selected lung region can be averaged over time, such as several minutes, to obtain a mean value of proton density (therefore eliminating small cyclic changes due to tidal ventilation). This mean value can then be compared to a previous mean value to determine temporal changes in pulmonary edema. Specifically, since most of the lung is gas, any change in mean detected signals will be due to changes in proton density or lung water (tissue and blood) fraction. In some cases, mean density measurements can be compared in the time range of every few hours in order to monitor changes in pulmonary edema.

Using similar methods, the portable magnetic resonance system 10 can be used as a non-invasive device to monitor pneumonia and the progression or decline of the disease during treatment. Furthermore, the portable magnetic resonance system 10 can assist in the diagnosis of pulmonary diseases in non-critical situations. For example, measuring local proton density as a function lung volume can provide a measure of regional lung compliance. An increase in lung compliance can be correlated with emphysema, while a decrease in lung compliance can be correlated with interstitial lung diseases.

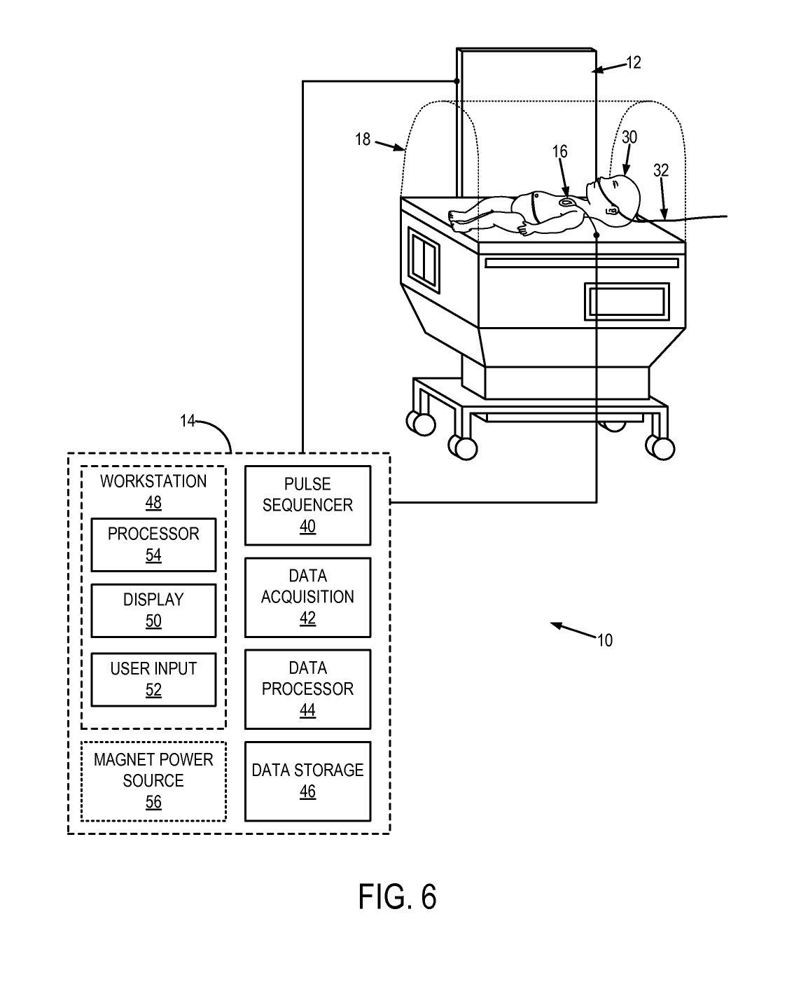

Referring now to FIG. 6, in one configuration of the portable magnetic resonance system 10, a hyperpolarized gas is provided to the subject 30, which may be an infant in a neonatal intensive care unit (“NICU”), and the portable magnetic resonance system 10 is operated to acquire magnetic resonance signals from the hyperpolarized gas. By way of example, the hyperpolarized gas may be helium-3, xenon-129, or the like. The hyperpolarized gas may be provided to the subject 30 by way of a cannula 32. In an alternative configuration, the hyperpolarized gas may be administered by a mask.

As discussed above, this configuration of the portable magnetic resonance system 10 has the advantage that by measuring magnetic resonance signals of a gas that is being inhaled and exhaled by the subject 30, a direct quantitative measurement of region ventilation is possible. On the other hand, this configuration requires the use of a hyperpolarized gas, which has the common drawbacks of using such a contrast agent.

The hyperpolarized gas can be provided, for example, by a laser polarizer that may be in a location adjacent to or remote from measurement environment. The use of hyperpolarized gas allows the portable magnetic resonance system 10 to perform sensitive measurements of lung ventilation without requiring a large magnetic field. In some instances, enriched xenon gas may be used, which can further increase SNR. It is also noted that, because hyperpolarized gases are benign, repeated longitudinal measurements can be made without harming the subject. The hyperpolarized gas can be provided to the cannula 32 or optional enclosure 18 prior to acquiring each measurement without significantly changing the fraction of inspired oxygen. This delivery method, together with the fact that the components of the portable magnetic resonance system 10 do not need to contact the subject, provide for a minimally invasive method of accurately assessing ventilatory function.

As infants in the NICU are often placed within plastic enclosures 18 or incubators, the portable magnetic resonance system 10 can be configured such that the magnet 12 is either outside or inside of the enclosure 18. If the magnet 12 is placed within the enclosure 18, the magnet 12 is sized to be sufficiently small so as to be fully enclosed within the enclosure 18. When the magnet 12 is positioned within the enclosure 18 it can be coupled to a mechanical assist mount (not shown) that may either be attached to the enclosure 18 or a stand-alone mount that penetrates the enclosure 18 through an entry hatch (not shown). If the magnet 12 is located outside of the enclosure 18, its size is not generally limited, thereby allowing for a magnet design that provides a larger homogeneous region. This is particularly useful in the Helmholtz pair configuration, since optimum field homogeneity for a Helmholtz pair is obtained when the Helmholtz coils are separated by a length of one radius. In some cases, the external magnet configuration may be considered a safer approach as the enclosure 18 can provide a physical barrier to objects that may be strongly attracted by the magnet 12.

When a smaller external magnet 12 is employed, the enclosure 18 can include a cover portion (not shown) to provide a shield between the magnet 12 and infant and to also allow close placement of the magnet 12 to the infant's chest. For example, the cover portion can include an indentation for proper magnet placement, and then the infant could be moved accordingly so that its chest is immediately adjacent to the indentation.

As discussed above, methods of the present invention can be used to provide measures of ventilation. Specifically, multiple measurements taken at different positions can be analyzed to determine the degree of regional ventilation heterogeneity. In addition to this relative measurement, absolute regional ventilation volumes or ratios of regional ventilation volumes relative to functional residual capacity (“FRC”) volume can be measured. A protocol for calibration of ventilated lung volumes can be realized according to the following logic, which considers both a single breath-hold experiment and a ventilator breathing experiment, in accordance with the methods described above, where a fixed amount of xenon-129 gas is delivered in each breath. As the hyperpolarized gas signal in the lung has inherently long T2* values at low fields (e.g., less than about 0.5 T) and the inhomogeneous field of the magnet 12 will artificially shorten T2* to values on the order of about one millisecond, the signal intensity observed is proportional to the amount of gas, polarization level, magnetization losses due to RF pulses, and T1. Both types of experiments can be calibrated accordingly to the procedures outlined below. For each experiment, multiple RF excitations are performed and either the free induction decay (FID) or the signal from a spin echo train is measured (processing of the signals will be the same for either case). As noted above, the spin echo train may be used to enhance SNR. It is well known to those skilled in the art that there are multiple ways to analyze magnetic resonance signals. In one example, a first way to define the signal to be processed is the initial measured amplitude of the FID. An alternative definition is the integrated intensity over the frequency spectrum for a specified bandwidth. For a particular experiment where there are n excitations, this processed signal is designated as S(n).

It is briefly noted that sample loading effects may be included when processing signals, however it may not be necessary since they are greatly reduced at low frequency due to the operation at low magnetic fields, in comparison to high field MRI. The effect of sample loading can be determined from reflection coefficient measurements of the loaded and unloaded RF coil.

For single breath-hold experiments, T1 and magnetization losses together can be measured from the signal decay curve from multiple small flip angle excitations, S(n). The polarization level can be measured independently before use. Thus, the corrected signal intensity, which is no longer dependent on n, will be proportional to the gas magnetization in the target field of view (“TFOV”). This corrected signal is designated as S_{c}. This measure by itself, when obtained from different target regions, can provide relative measures of regional ventilation. To ascertain the volume that is probed by a specific magnet geometry, TFOV can be determined from prior field mapping measurements. The calibration constant K that allows conversion of S_{c} to absolute volume can be determined from a phantom experiment and is given by K=S_{c}(phantom)/TFOV. To ensure the entire TFOV is being interrogated, the phantom can include a volume larger than the TFOV.

For continuous breathing experiments, consider that a small amount of xenon-129 magnetization is injected during each breath (A_{Xe}) as a bolus from the hyperpolarized gas reservoir, where A_{Xe} has units of magnetization. For each inhalation of a tidal volume (“VT”), it is assumed that uniform mixing occurs by the end of inspiration. Then upon expiration of VT, some of the xenon magnetization is exhaled. After n breaths, it can be shown by induction that the magnetization concentration (units of magnetization per unit volume) of xenon-129 gas, is given by

$$[ {}^{129} X e ] ( n ) = \frac{{A}_{Xe} α ( 1 - {α}^{n} )}{FRC ( 1 - α )} ; \qquad ( 7 )$$

where FRC is the functional residual capacity and α=FRC/(FRC+VT). For a sufficiently large number of breaths (n→∞), the steady state concentration, [¹²⁹Xe](∞), will be given by

$$[ {}^{129} X e ] ( ∞ ) = \frac{{A}_{Xe} α}{FRC ( 1 - α )} . \qquad ( 8 )$$

If the loss of signal due to T1 and RF depletion is to be included, the easiest solution is found with the assumption of uniform time intervals of the breathing cycle that are synchronized with the RF pulse repetition time (“TR”). For this case, let β represent the fractional signal loss that occurs through RF depletion and. T1 decay during a TR. Given this, α can be replaced with αβ in the above equations. It is noted that, for simplicity, the case where RF pulses are synchronized with breathing is herein described. However, an average RF depletion loss can still be calculated even if the RF pulses are not synchronized with breathing. The steady state signal measured, S_{SS}, is given by

$${S}_{SS} = K \frac{TFOV}{{A}_{Xe}} {V}_{EE} [ {}^{129} X e ] ( ∞ ) ; \qquad ( 9 )$$

where V_{EE} is the volume of hyperpolarized gas within the region-of-interest at end expiration and K is the calibration constant described above for the breath-hold experiment. Substituting Equation (8) into Equation (9) and rearranging terms provides the following:

$$\frac{{V}_{EE}}{FRC} = \frac{{S}_{SS}}{K \cdot TFOV} \frac{1 - αβ}{αβ} . \qquad ( 10 )$$

Using Equation (7), αβ can be obtained from a fit with respect to n to the initial rise of the xenon-129 signal. Thus, from a measurement of the steady state magnetization concentration signal, S_{SS}, a measurement of the ratio of the regional end expiratory volume to the total lung functional residual capacity can be obtained. If it is also possible to perform a brief breath-hold experiment in addition to the continuous breathing experiment, actual values of V_{EE} and FRC can be obtained. From the static breath-hold experiment, measurements from a small phantom containing a known volume of hyperpolarized gas can be used as an absolute calibration between signal intensities and regional gas volume. Longitudinal measurements in the lungs of a subject can be compared with the measurements from the phantom to effectively remove any uncertainty due to changing polarization levels in the inspired gas. It is also noted that, since there will only be a detected signal if the hyperpolarized gas ventilates the particular region, signal contributions from surrounding tissues do not need to be subtracted out during processing. Although xenon is soluble in tissue, the dissolved phase/tissue signal is negligible compared to the gas phase ventilation signal.

Having described different configurations of the portable magnetic resonance system 10 of the present invention, attention is now drawn to a general method for operating the portable magnetic resonance system 10 and for producing quantitative metrics, such as regional lung ventilation and lung density, from magnetic resonance signals acquired with the portable magnetic resonance system 10.

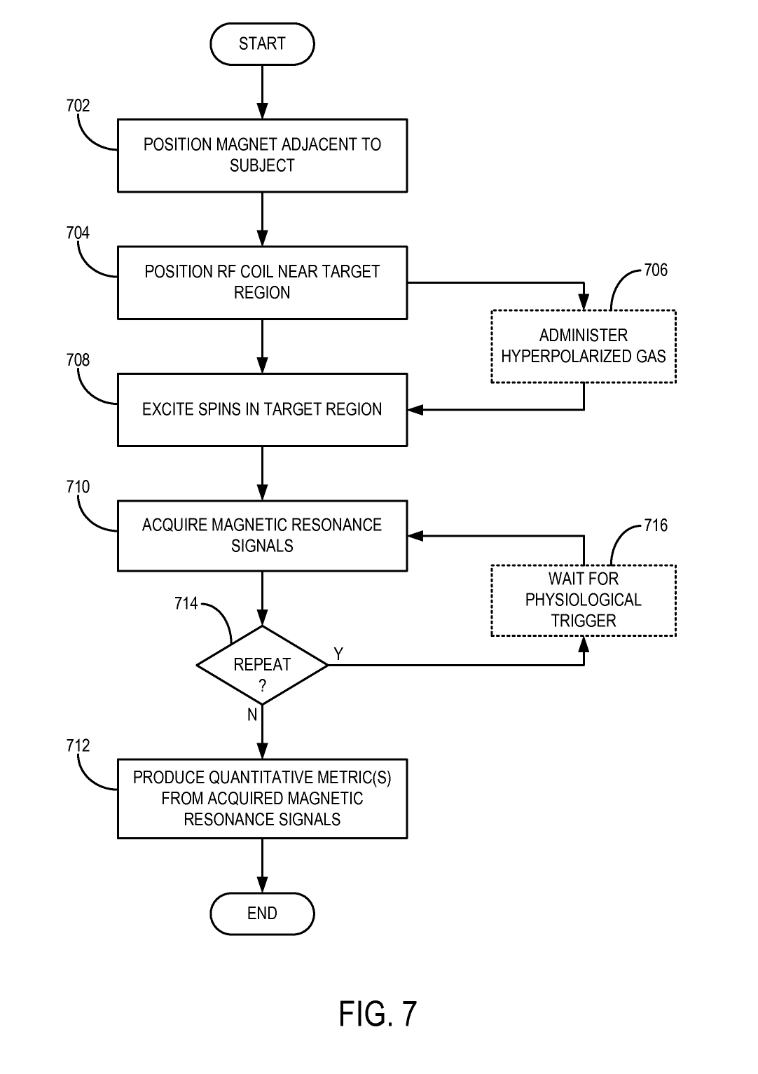

In use, the portable magnetic resonance system 10 acts as a portable, magnetic resonance spectrometer capable of detecting the presence of water protons associated with lung tissue or of a hyperpolarized gas after it has been inhaled in a region of a subject's lung. Referring now to FIG. 7, a flowchart setting forth the steps of an example of a method for producing a quantitative metric indicative of a property of a subject's lung using the portable magnetic resonance system 10 of the present invention is illustrated. The method generally includes positioning the magnet 12 of the portable magnetic resonance system 10 adjacent to the subject to produce an external magnetic field, B₀, that is homogeneous in a target region that is external to the structure of the magnet 12, as indicated at process block 702. In particular, the magnet 12 is positioned such that the external magnetic field projects into the target region of the subject's lung. Following this, an RF coil that forms a part of the RF coil assembly 16 is positioned near the target region, as indicated at process block 704. Optionally, a bolus of hyperpolarized gas may be introduced into the subject's lung through inhalation, as indicated at process block 706, before magnetic resonance signals are acquired from the subject.

Next, as indicated at step 708, spins are excited in the target region by producing an appropriately tuned RF excitation pulse with the RF coil. If the portable magnetic resonance system 10 is being operated to acquire magnetic resonance signals from water protons in lung tissue and blood, then the RF excitation pulse is tuned to the Larmor frequency of hydrogen. If, however, a hyperpolarized gas has been provided to the subject, then the RF excitation pulse is tuned to the Larmor frequency of the hyperpolarized gas used, such that magnetic resonance signals will be acquired from the hyperpolarized gas. Magnetic resonance signals responsive to the RF excitation are then acquired, as indicated at step 710. From the acquired magnetic resonance signals, a quantitative metric indicative of a property of the subject's lung is calculated, as indicated at step 712. By way of example, the quantitative metric may be region lung ventilation or lung density. Optionally, an image indicative of the quantitative metric may be produced by making measurements at multiple positions. In such an image, each individual measurement would be represented as a single voxel in the image; thus, the voxel size in this image would be the size of the region-of-interest from which the lung property measurement is made. For example, the image whose voxel values are determined by the measured lung ventilation or lung density may be produced. However, because the portable magnetic resonance system 10 produces measurements of regional lung ventilation or lung density with a spatial resolution that is equivalent to the region-of-interest from which measurements are obtained, such images would have a limited number of voxels or very coarse spatial resolution as compared to traditional magnetic resonance images.

As indicated at decision block 714, these steps can be repeated. For example, magnetic resonance signals can be acquired at different inhalation volumes, different ventilator pressures, or synchronously with other physiological parameters, and density measurements can be compared across these different parameters. Thus, when additional signal acquisitions are performed, they may optionally be triggered by a physiological trigger, such as one of the aforementioned parameters, as indicated at step 716. For example, relative changes in the intensity or amplitude of the free induction decay (“FID”) signal across these different volumes will reflect changes in proton density. In particular, signal data acquisition can be gated in accordance with a ventilator providing ventilation to the subject, such as at full inspiration and at full expiration. Gating also eliminates requirements of a subject to hold their breath during data acquisition. Comparison of proton density measurements between inspiration and expiration can then provide a quantitative measure of how well the lung is ventilated. In some cases, comparisons can be evaluated based on known change ratios in lung density. For example, in healthy subjects, lung density is known to change by a factor of four when going from residual volume (“RV”) to total lung capacity (“TLC”). There is also a notable change in lung density in healthy subjects between RV and functional residual capacity (“FRC”), as well as between FRC and TLC. In addition, changes in proton density over time can be compared to determine increase or decrease of pulmonary edema. The subject's lung density or ventilation in a particular region can serve as a known control that can be used for comparison to other regions in the subject's lung.

In some implementations of the portable magnetic resonance system 10, a simulation program can be used, for example at the workstation 48, with a magnet positioning tool (not shown) in order to automatically adjust magnet positioning (such as adjusting a distance between magnets 12 or the relative angles of the magnets 12 through rotation) during use of the portable magnetic resonance system 10. Because different patients have different amounts of muscle, fat, and other tissue on the surface of their body, the distance between the outside surface of the patient and the beginning of the lung varies. Thus, increasing the distance between magnets 12 can increase the distance of the target region from the magnet surfaces, and vice versa. In some instances, the distance of the target region from the magnet surface required to reach the lung parenchyma can be determined by probing the depth at which the lung parenchyma begins using a ultrasound probe (specifically, to demarcate the boundary between intercostal and pleural soft tissue and the lung proper). The magnet positioning tool can then be used to position an outer edge of the detection region to reach the ultrasound probed depth so that the entire detection region lies within the lung. In combination with this ultrasound method, a basic method for determining when the detection region is in the thorax is to adjust the magnets 12 (for example, by moving the magnets closer to the patient's chest) while continuously monitoring SNR. A sudden drop in SNR can indicate that the detection region is in the thorax.

One issue to be considered when designing the portable magnetic resonance system 10 is that despite the RF coils being at low frequency (that is, less than about two MHz), coupling of extraneous RF noise can be significant, and therefore can result in significant decreases in SNR. The nature of the noise typically includes both broadband components (that is, white noise components) and narrowband components (that is, spurious noise components).

In a typical MRI scanner, these noise sources are eliminated by placing the MRI system in an RF shielded room. However, this may be inconvenient and impractical for environments using a portable magnetic resonance system 10, such as a NICU, ICU, field hospital, or battlefield evacuation vehicle. Accordingly, the RF coil assembly 16 of the portable magnetic resonance system 10 can utilize noise cancelling RF coils. It is also contemplated that some NICU or ICU environments may employ a dedicated RF shielded room for the portable magnetic resonance system 10, thereby removing the need for additional RF shielding techniques and specifically delineating an magnetic resonance safe zone. In these cases, subjects may be moved into the RF shielded room while on a portable ventilator.

In one example, noise cancelling coils may be produced as a single figure eight coil. In another configuration, the noise cancelling coils may be produced as two coils that are 180 degrees out of phase with each other. In some situations, multiple sets of coils may be used. In analog terms, this subtraction can be performed by wiring the coils appropriately in series (such as in the figure eight coil configuration) or combining the outputs from the two coils after appropriate scaling and phase shifting is performed. Such analog methods may be able to achieve a reduction to about a one percent level (a 40 dB reduction). As this may not be sufficient for maximum noise cancellation, digital methods, which allow the use of post processing algorithms, can also be used to achieve better noise cancellation performance.

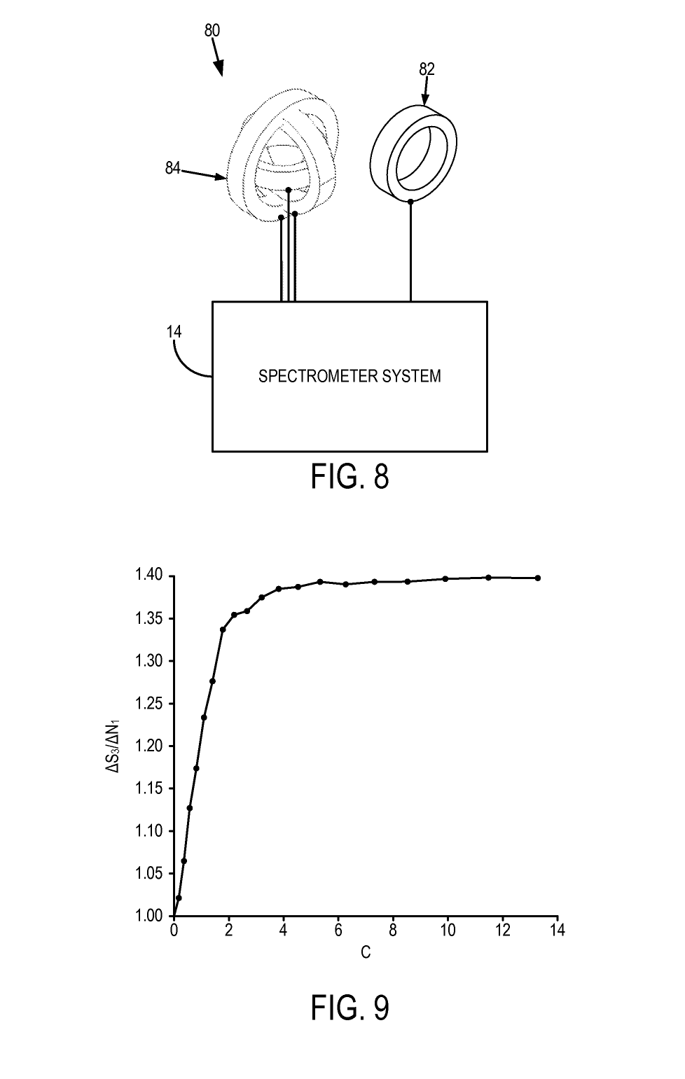

In another configuration representative of a “digital” noise cancellation, two coil sets are interfaced to a multichannel spectrometer system, as illustrated in FIG. 8. One coil termed the “signal coil” 82 detects magnetic resonance signals as well as environmental noise, while the other coil termed the “noise reference coil” 84 detects substantially only environmental noise. The noise reference coil 84 illustrated in FIG. 8 includes three orthogonal coils. In principal, a subtraction of the signal received on the signal coil from the signal received on the noise reference coil could be used to eliminate the unwanted interference. This subtraction can take place within the processing system of the spectrometer. In one example of this multiple coil set configuration, two coil sets are employed for the noise cancellation system. The first coil set is the signal coil set including a single coil or plurality of coils used to detect the magnetic resonance signal response. The second coil set is the noise reference coil set, which includes a single coil or a plurality of coils used to detect the ambient noise from the environment. Each individual coil is sampled simultaneously by a multichannel electronics system or spectrometer. In another configuration, the noise reference coil may include two or more coils whose coil axes are oriented along orthogonal spatial directions.

In accordance with one aspect of the invention, the following algorithm can be implemented to determine a transfer function between reference noise measured from the noise reference coil and a measured signal measured from the signal coil that optimizes active noise cancellation. Considering the signals from a signal coil and a noise reference coil, a multichannel detector can be used to simultaneously capture noise signals with magnetic resonance signals. In this case, S is the total signal measured (that is, in voltage) from a coil inclusive of the magnetic resonance signal, Johnson noise, environmental white noise, and environmental spurious noise, The magnetic resonance signal is represented by F. Johnson noise (Nᵤ) is always uncorrelated between coils. On the other hand environmental noise (N_{c}) will always be correlated among coils. For convenience, white and spurious environmental noise may be grouped together. Thus, in a two coil arrangement the total signal from the signal coil is given by

S₁=F+Nᵤ₁+N_{c} (11);

while the signal from the noise reference coil is given by

S₂=Nᵤ₂+βN_{c} (12);

and the processed signal with noise subtraction is given by

S₃=S₁−αS₂ (13);

where it is assumed that the correlated noise is scaled (amplitude and phase) differently between the two coils with a complex factor β. S₃ is the desired signal obtained by scaling S₂ by a complex scaling factor α. Without loss in generality, S may be a function of time or frequency. Reducing the noise is then a least squares minimization problem whereby a solution for a is obtained by minimizing A.

A=Σ|S₁ᵢ−αS₂ᵢ|² (14).

Cross-terms (Nᵤ×N_{c}, Nᵤ₁×Nᵤ₂) generated in the evaluation of A will be zero provided that all components (Nᵤ and N_{c}) are uncorrelated. This leads to expressions for the magnitude (ρ) and phase (φ) of α=pe^{iφ} given by:

$$\begin{matrix}ρ = \frac{\sqrt{{\sum }_{i}^{ } {S}_{1 i} {S}_{2 i}^{*} {\sum }_{i}^{ } {S}_{1 i}^{*} {S}_{2 i}}}{{\sum }_{i}^{ } {S}_{1 i}^{*} {S}_{2 i}} ; & ( 15 ) \\ φ = - \frac{i}{2} ln ( \frac{{\sum }_{i}^{ } {S}_{1 i} {S}_{2 i}^{*}}{{\sum }_{i}^{ } {S}_{1 i}^{*} {S}_{2 i}} ) . & ( 16 )\end{matrix}$$

Considering the above, α is not equal to 1/β, as might be expected. Furthermore, as the correlated noise increases from zero, the magnitude ranges from 0 to 1. Another observation is that the above algorithm may work if a signal or spurious noise is present as the cross-terms (F×Nᵤ, Nᵤ×N_{c}) may be sufficiently small as they still involve a random factor. The exception may be when there is correlation between the signal and a spurious noise component. This suggests that at the very least a calibration scheme will work. Thus, α may be calibrated by measurements taken before the acquisition of the magnetic resonance signal, and then after calibration α is applied in a real-time calculation of S₃. In addition, in some implementations, a frequency dependent complex factor or noise reference coils oriented in different directions (for example, to fully characterize the environmental noise) may be implemented. Accordingly, in such implementations, calculations may be performed in the frequency domain.

Theoretically, if two coils (specifically, signal and noise reference coils) of equal construction are used, then the Johnson noise power is always doubled. This would suggest that noise cancellation suffers a \sqrt{2} loss in SNR. However, the following two methods suggest that this is not true. First, the deterministic algorithm described above scales as a function of the intensity of the environmental noise. As N_{c}→∞, Johnson noise power in S₃ doubles, but as N_{c}→0, α=0 and Johnson noise power is not doubled. FIG. 9 illustrates a numerical calculation of the noise penalty factor (in terms of ΔS₃/ΔN₁) as a function of the ratio of the correlated random noise to the uncorrelated random noise. As shown in FIG. 9, noise penalty factor varies from 1 to \sqrt{2} as the amount of correlated noise increases. A second method is to use a noise reference coil that is larger in cross sectional area than the signal coil. Thus, the ratio of the environmental noise to Johnson noise in the noise reference coil will be larger and the Johnson noise contribution from the noise reference coil will proportionately be reduced in S₃.

If for practical reasons a large coil is difficult to implement in a portable MR device, extra coils can be added to achieve the same effect. However, it is noted that a larger coil differs in terms of power in comparison to the multiple coil design. For example, if a large coil has an area four times greater than the signal coil, the environmental noise power will increase eight-fold, while the Johnson noise power remains the same. Thus, in calculating the contribution in the corrected signal (S₃), the Johnson noise power has only increased by one eighth. On the other hand, if four noise reference coils are used, each equal in area to the signal coil, the combined environmental noise power only increases a factor of four over the combined Johnson noise power. In addition, it is noted that, when the signal and noise reference coils are of different sizes or construction, the bandwidth of the two coils will be different. As these bandwidths will be known, a first step prior to the above noise cancelation algorithm can be included where the signal from each coil will be deconvoluted by the bandwidth response.

Accordingly, using the above noise cancellation algorithms with two magnetic resonance detectors (specifically, the signal coil and the noise reference coil), the portable magnetic resonance system 10 can be used without the need for an RF shielded room. These techniques can further be applied to other portable magnetic resonance devices, as well as other electronic RF instrumentation that is sensitive to environmental RF noise.

The present invention has been described in terms of one or more preferred embodiments, and it should be appreciated that many equivalents, alternatives, variations, and modifications, aside from those expressly stated, are possible and within the scope of the invention.

1. A portable magnetic resonance system configured to acquire magnetic resonance signals generated in a region-of-interest in a subject's lung and to calculate therefrom a quantitative metric indicative of a property of the subject's lung, comprising:
a magnet sized to be positioned proximate to a subject and configured to generate a magnetic field that is substantially homogeneous in a region-of-interest positioned at a distance from a surface of the magnet that is sufficiently large so as to position the region-of-interest in the subject's lung;
a radio frequency (RF) coil assembly including at least one RF coil sized to be positioned proximate to the region-of-interest and configured to apply an RF field to the region-of-interest and to receive magnetic resonance signals therefrom;
a spectrometer system in communication with the RF coil assembly and programmed to:
direct the RF coil assembly to produce an RF field in the region-of-interest at a Larmor frequency such that spins resonant with the Larmor frequency in the region-of-interest are excited;
direct the RF coil assembly to receive magnetic resonance signals produced in the region-of-interest in response to the applied RF field; and
compute from the acquired magnetic resonance signals a quantitative metric indicative of a characteristic of the subject's lung in the region-of-interest.

2. The portable magnetic resonance system as recited in claim 1 in which the quantitative metric computed by the spectrometer system is at least one of lung ventilation and lung density.

3. The portable magnetic resonance system as recited in claim 1 in which the magnet is sized such that its thickness is less than both its width and its length.

4. The portable magnetic resonance system as recited in claim 1 further comprising means for providing a hyperpolarized gas to the subject.

5. The portable magnetic resonance system as recited in claim 1 further comprising an enclosure sized to receive the subject.

6. The portable magnetic resonance system as recited in claim 5 in which the magnet is sized to be contained within the enclosure.

7. The portable magnetic resonance system as recited in claim 1 in which the magnet is at least one of a permanent magnet and an electromagnet.

8. The portable magnetic resonance system as recited in claim 7 in which the magnet is a permanent magnet that is configured as a ferro-refraction magnet.

9. The portable magnetic resonance system as recited in claim 8 further comprising a shield that is positioned on substantially only one side of the magnet.

10. The portable magnetic resonance system as recited in claim 7 in which the magnet is configured as at least one of a monohedral magnet, a planar magnet, and a Helmholtz-pair magnet.

11. The portable magnetic resonance system as recited in claim 1 in which the magnet includes a first pole and a second pole that are positioned opposite each other about the at least one RF coil.

12. The portable magnetic resonance system as recited in claim 11 further comprising an enclosure that is sized to contain the first pole, the second pole, and the at least one RF coil.

13. The portable magnetic resonance system as recited in claim 12 in which the enclosure is sized to be positioned between a subject and a bed.

14. The portable magnetic resonance system as recited in claim 1 in which the at least one RE coil comprises at least one transmit RF coil and at least one receive RF coil.

15. The portable magnetic resonance system as recited in claim 14 in which the RF coil assembly includes one transmit RF coil and one receive RF coil, and in which the transmit RE coil and receive RE coil are concentric.

16. The portable magnetic resonance system as recited in claim 14 in which the RE coil assembly includes one transmit RF coil and a plurality of receive RF coils.

17. The portable magnetic resonance system as recited in claim 16 in which the one transmit RF coil includes a Helmholtz pair and the plurality of receive RE coils are positioned within the Helmholtz pair.

18. The portable magnetic resonance system as recited in claim 1 in which the RF coil assembly includes at least one signal RF coil configured to receive magnetic resonance signals and at least one noise reference RE coil configured to receive substantially only signals indicative of environmental noise.

19. The portable magnetic resonance system as recited in claim 18 in which the spectrometer system is programmed to significantly reduce noise in the received magnetic resonance signals using the received signal that is indicative of substantially only environmental noise.

20. (canceled)

21. A method for actively cancelling electronic noise in a nuclear magnetic resonance device, the steps of the method comprising:
a) acquiring with a first radio frequency (RF) coil, a signal that contains a magnetic resonance signal and a noise signal;
b) acquiring with a second RF coil, a noise reference signal that contains substantially only environmental noise;
c) calculating a scaling factor that scales noise that is correlated in the first RF coil and the second RF coil;
d) producing a scaled noise signal by applying the scaling factor calculated in step c) to the noise reference signal acquired in step h); and
e) producing a substantially noise-free signal by subtracting the scaled noise signal from the signal acquired in step a).

22-28. (canceled)
